(19) United States
(12) Patent Application Publication
(10) Pub. No.: US 2024/0016845 A1
(43) Pub. Date: Jan. 18, 2024

(54) OVERCOMING TNF-ALPHA BLOCKADE RESISTANCE IN RHEUMATOID ARTHRITIS BY REGENERATIVE T REGULATORY CELL THERAPY

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Niceville, FL (US)

(73) Assignee: CREATIVE MEDICAL TECHNOLOGIES, INC., Phoenix, AZ (US)

(21) Appl. No.: 18/348,288

(22) Filed: Jul. 6, 2023

Related U.S. Application Data
(60) Provisional application No. 63/389,091, filed on Jul. 14, 2022.

(51) Int. Cl. A61K 35/17; C12N 5/0783

(52) U.S. Cl. CPC A61K 35/17; C12N 5/0637; C12N 2502/45

(57) ABSTRACT

Treatment of rheumatoid arthritis has been revolutionized by the introduction of TNF-alpha blockers, which represent a 40 billion dollar annual market. Unfortunately, a significant proportion of patients fail to respond to these treatments. The current invention provides the use of stem cell conditioned T cells to overcome resistance to TNF-alpha blockers by concurrently providing a source of immune modulatory and regenerative cells that synergize with TNF-alpha blockers.

### CROSS-REFERENCE TO RELATED APPLICATIONS

This application is a non-provisional of and claims priority to U.S. Provisional Application Ser. No. 63/389,091, titled “Overcoming TNF-alpha Blockade Resistance in Rheumatoid Arthritis by Regenerative T regulatory Cell Therapy”, filed Jul. 14, 2022, which is hereby incorporated by reference in its entirety.

### FIELD OF THE INVENTION

The invention belongs to the field of treating rheumatoid arthritis through the use of regenerative T-cells.

### BACKGROUND

It is known that Rheumatoid arthritis (RA) is an inflammatory autoimmune disorder which is inflammatory in nature and affects up to 1% of the population. This condition is characterized by irreversible joint damages, with disability and ultimately accelerated atherosclerotic cardiovascular and coronary heart disease. Chronic infiltration of the joints by activated immune competent cells including macrophages, T and B cells, together with synovial tissue hyperplasia, leads to cartilage and bone destruction after several years. Although the causes of RA are not fully understood, numerous studies indicate that cytokines are critical in the processes that cause inflammation and joint destruction, TNF-alpha being definitively the prominent one. Currently, in clinical practice, if disease activity cannot be controlled with conventional disease modifying anti-rheumatic drugs (DMARD), anti-TNF biotherapies are used. Although a major breakthrough has emerged in the management of RA patients with TNF-alpha blockade, it is not curative and its effects are only partial. In addition, non-responders to the treatment are common and loss of effect are observed. Up to 40% of patients are refractory to TNF-alpha blockade. Taking into account the cost of these treatments, the persisting doubts about potential long term adverse events and the availability of other efficient biotherapies in the treatment of RA, selection, development of means of increasing efficacy of TNF-alpha blockers, as well as overcoming resistance to these agents is a critical unmet need.

### SUMMARY

Preferred embodiments are directed to methods of overcoming resistance to TNF-alpha blockade in a patient with rheumatoid arthritis comprising the steps of: a) obtaining a sample of peripheral blood mononuclear cells from said patient; b) exposing said cells to conditioned media from a regenerative cell population; c) generating said conditioned media of “b” through activating said regenerative cell population; d) obtaining said patient peripheral blood mononuclear cells or isolated component cells thereof; e) optionally expanding said cells ex vivo; and f) administering said heterogenous or said homogeneous cell population of “e” into said patient in need of therapy.

Preferred methods are drawn to embodiments wherein said peripheral blood mononuclear cells are taken from a patient after exposure to mobilization therapy.

Preferred methods are drawn to embodiments wherein said mobilization therapy comprises administration to the patient a sufficient amount of VEGF to induce mobilization of stem cell progenitors into circulation.

Preferred methods are drawn to embodiments wherein said mobilization therapy comprises administration to the patient a sufficient amount of SDF-1 blocking antibody to induce mobilization of stem cell progenitors into circulation.

Preferred methods are drawn to embodiments wherein said mobilization therapy comprises administration to the patient a sufficient amount of G-CSF to induce mobilization of stem cell progenitors into circulation.

Preferred methods are drawn to embodiments wherein said mobilization therapy comprises administration to the patient a sufficient amount of M-CSF to induce mobilization of stem cell progenitors into circulation.

Preferred methods are drawn to embodiments wherein said mobilization therapy comprises administration to the patient a sufficient amount of GM-CSF to induce mobilization of stem cell progenitors into circulation.

Preferred methods are drawn to embodiments wherein said mobilization therapy comprises administration to the patient a sufficient amount of Mozobil to induce mobilization of stem cell progenitors into circulation.

Preferred methods are drawn to embodiments wherein immune cells are isolated from peripheral blood mononuclear cells.

Preferred methods are drawn to embodiments wherein said immune cells are T cells.

Preferred methods are drawn to embodiments wherein said T cells are CD4 cells.

Preferred methods are drawn to embodiments wherein said T cells are CD8 cells.

Preferred methods are drawn to embodiments wherein said T cells are NKT cells.

Preferred methods are drawn to embodiments wherein said T cells are gamma delta T cells.

Preferred methods are drawn to embodiments wherein said T cells are Th2 cells.

Preferred methods are drawn to embodiments wherein said Th2 cells have a proclivity to produce more interleukin-4 than interferon gamma upon stimulation via CD3.

Preferred methods are drawn to embodiments wherein said Th2 cells express GATA-3.

Preferred methods are drawn to embodiments wherein said Th2 cells express IRF-4.

Preferred methods are drawn to embodiments wherein said Th2 cells express STAT5.

Preferred methods are drawn to embodiments wherein said Th2 cells express STAT6.

Preferred methods are drawn to embodiments wherein said Th2 cells express CCR3.

Preferred methods are drawn to embodiments wherein said Th2 cells express CCR4.

Preferred methods are drawn to embodiments wherein said Th2 cells express CCR8.

Preferred methods are drawn to embodiments wherein said Th2 cells express CXCR4.

Preferred methods are drawn to embodiments wherein said Th2 cells express interleukin-4 receptor.

Preferred methods are drawn to embodiments wherein said Th2 cells express interleukin-33 receptor.

Preferred methods are drawn to embodiments wherein said T cells are Th9 cells.

Preferred methods are drawn to embodiments wherein said Th9 cell produces interleukin-9.

Preferred methods are drawn to embodiments wherein said Th9 cell expresses IRF4.

Preferred methods are drawn to embodiments wherein said Th9 cell expresses PU.1.

Preferred methods are drawn to embodiments wherein said Th9 cell secretes CCL17.

Preferred methods are drawn to embodiments wherein said Th9 cell secretes CCL22.

Preferred methods are drawn to embodiments wherein said Th9 cell secretes IL-10.

Preferred methods are drawn to embodiments wherein said Th9 cell expresses TGF-beta receptor II.

Preferred methods are drawn to embodiments wherein said T cell is a follicular helper T cell.

Preferred methods are drawn to embodiments wherein said follicular helper T cell expresses bcl-6.

Preferred methods are drawn to embodiments wherein said follicular helper T cell expresses c-maf.

Preferred methods are drawn to embodiments wherein said follicular helper T cell expresses stat-3.

Preferred methods are drawn to embodiments wherein said follicular helper T cell secretes CXCL-13.

Preferred methods are drawn to embodiments wherein said follicular helper T cell secretes interferon gamma.

Preferred methods are drawn to embodiments wherein said follicular helper T cell secretes interleukin-4.

Preferred methods are drawn to embodiments wherein said follicular helper T cell secretes IL-10.

Preferred methods are drawn to embodiments wherein said follicular helper T cell secretes IL-17A.

Preferred methods are drawn to embodiments wherein said follicular helper T cell secretes IL-17F.

Preferred methods are drawn to embodiments wherein said follicular helper T cell secretes IL-21.

Preferred methods are drawn to embodiments wherein said follicular helper T cell expresses BTLA-4.

Preferred methods are drawn to embodiments wherein said follicular helper T cell secretes CD40 ligand.

Preferred methods are drawn to embodiments wherein said follicular helper T cell expresses CD57.

Preferred methods are drawn to embodiments wherein said follicular helper T cell expresses CD84.

Preferred methods are drawn to embodiments wherein said follicular helper T cell expresses CXCR-4.

Preferred methods are drawn to embodiments wherein said follicular helper T cell expresses CXCR-5.

Preferred methods are drawn to embodiments wherein said follicular helper T cell expresses ICOS.

Preferred methods are drawn to embodiments wherein said follicular helper T cell expresses IL-6 receptor.

Preferred methods are drawn to embodiments wherein said follicular helper T cell expresses IL-21 receptor.

Preferred methods are drawn to embodiments wherein said follicular helper T cell expresses CD10.

Preferred methods are drawn to embodiments wherein said follicular helper T cell expresses OX40.

Preferred methods are drawn to embodiments wherein said follicular helper T cell expresses PD-1.

Preferred methods are drawn to embodiments wherein said follicular helper T cell expresses CD150.

Preferred methods are drawn to embodiments wherein said regenerative cell population is a pluripotent stem cell.

Preferred methods are drawn to embodiments wherein said pluripotent stem cell is an embryonic stem cell.

Preferred methods are drawn to embodiments wherein said pluripotent stem cell is an inducible pluripotent stem cell.

Preferred methods are drawn to embodiments wherein said pluripotent stem cell is an inducible pluripotent stem cell generated by viral transfection.

Preferred methods are drawn to embodiments wherein said pluripotent stem cell is an inducible pluripotent stem cell generated through the use of miRNAs.

Preferred methods are drawn to embodiments wherein said pluripotent stem cell is an inducible pluripotent stem cell is generated through addition of small molecules.

Preferred methods are drawn to embodiments wherein said pluripotent stem cell is a parthenogenic stem cell.

Preferred methods are drawn to embodiments wherein said pluripotent stem cell is generated by somatic cell nuclear transfer.

Preferred methods are drawn to embodiments wherein said regenerative cell is a mesenchymal stem cell.

Preferred methods are drawn to embodiments wherein said mesenchymal stem cell is plastic adherent.

Preferred methods are drawn to embodiments wherein said mesenchymal stem cell expresses CD90 and NANOG−.

Preferred methods are drawn to embodiments wherein said mesenchymal stem cell expresses CD74 and SSEA-4.

Preferred methods are drawn to embodiments wherein said mesenchymal stem cell expresses CD73.

Preferred methods are drawn to embodiments wherein said mesenchymal stem cell expresses CD105.

Preferred methods are drawn to embodiments wherein said mesenchymal stem cell expresses CD37.

Preferred methods are drawn to embodiments wherein said mesenchymal stem cell expresses Fas ligand.

Preferred methods are drawn to embodiments wherein said mesenchymal stem cell expresses CD150.

Preferred methods are drawn to embodiments wherein said mesenchymal stem cell expresses HLA-G

Preferred methods are drawn to embodiments wherein said mesenchymal stem cell expresses ILT3.

Preferred methods are drawn to embodiments wherein said mesenchymal stem cell expresses ILT4

Preferred methods are drawn to embodiments wherein said mesenchymal stem cell expresses indolamine-2,3-deoxygenase.

Preferred methods are drawn to embodiments wherein said T cell is a Th22 cell.

Preferred methods are drawn to embodiments wherein said Th22 cell secretes IL-10.

Preferred methods are drawn to embodiments wherein said Th22 cell secretes IL-13.

Preferred methods are drawn to embodiments wherein said Th22 cell secretes FGF-1.

Preferred methods are drawn to embodiments wherein said Th22 cell secretes FGF-2.

Preferred methods are drawn to embodiments wherein said Th22 cell secretes FGF-5.

Preferred methods are drawn to embodiments wherein said Th22 cell secretes IL-21.

Preferred methods are drawn to embodiments wherein said Th22 cell secretes IL-22.

Preferred methods are drawn to embodiments wherein said Th22 cell expresses AHR.

Preferred methods are drawn to embodiments wherein said Th22 cell expresses batf.

Preferred methods are drawn to embodiments wherein said Th22 cell expresses STAT-3

Preferred methods are drawn to embodiments wherein said Th22 cell expresses CCR4.

Preferred methods are drawn to embodiments wherein said Th22 cell expresses CCR6.

Preferred methods are drawn to embodiments wherein said Th22 cell expresses CCR10.

Preferred methods are drawn to embodiments wherein said Th22 cell expresses IL-6 receptor.

Preferred methods are drawn to embodiments wherein said Th22 cell expresses TGF-beta receptor II.

Preferred methods are drawn to embodiments wherein said Th22 cell expresses TNF receptor 1.

Preferred methods are drawn to embodiments wherein said T cell is a T regulatory cell.

Preferred methods are drawn to embodiments wherein said T regulatory cell expresses foxp-3.

Preferred methods are drawn to embodiments wherein said T regulatory cell expresses Helios.

Preferred methods are drawn to embodiments wherein said T regulatory cell expresses STAT5.

Preferred methods are drawn to embodiments wherein said T regulatory cell expresses CD5.

Preferred methods are drawn to embodiments wherein said T regulatory cell expresses CD25.

Preferred methods are drawn to embodiments wherein said T regulatory cell expresses CD39.

Preferred methods are drawn to embodiments wherein said T regulatory cell expresses CD105.

Preferred methods are drawn to embodiments wherein said T regulatory cell expresses IL-7 receptor.

Preferred methods are drawn to embodiments wherein said T regulatory cell expresses CTLA-4.

Preferred methods are drawn to embodiments wherein said T regulatory cell expresses folate receptor.

Preferred methods are drawn to embodiments wherein said T regulatory cell expresses CD223

Preferred methods are drawn to embodiments wherein said T regulatory cell expresses LAP.

Preferred methods are drawn to embodiments wherein said T regulatory cell expresses GARP.

Preferred methods are drawn to embodiments wherein said T regulatory cell expresses Neuropilin.

Preferred methods are drawn to embodiments wherein said T regulatory cell expresses CD134.

Preferred methods are drawn to embodiments wherein said T regulatory cell expresses CD62 ligand.

Preferred methods are drawn to embodiments wherein said T regulatory cell secretes IL-10.

Preferred methods are drawn to embodiments wherein said T regulatory cell secretes TGF-alpha.

Preferred methods are drawn to embodiments wherein said T regulatory cell secretes TGF-beta.

Preferred methods are drawn to embodiments wherein said T regulatory cell secretes soluble TNF receptor p55.

Preferred methods are drawn to embodiments wherein said T regulatory cell secretes soluble TNF receptor p75.

Preferred methods are drawn to embodiments wherein said T regulatory cell secretes IL-2.

Preferred methods are drawn to embodiments wherein said T regulatory cell secretes soluble HLA-G.

Preferred methods are drawn to embodiments wherein said T regulatory cell secretes soluble Fas ligand.

Preferred methods are drawn to embodiments wherein said T regulatory cell secretes IL-35.

Preferred methods are drawn to embodiments wherein said T regulatory cell secretes VEGF.

Preferred methods are drawn to embodiments wherein said T regulatory cell secretes HGF.

Preferred methods are drawn to embodiments wherein said T regulatory cell secretes FGF1.

Preferred methods are drawn to embodiments wherein said T regulatory cell secretes FGF2.

Preferred methods are drawn to embodiments wherein said T regulatory cell secretes FGF5.

Preferred methods are drawn to embodiments wherein said T regulatory cell secretes Galectin 1.

Preferred methods are drawn to embodiments wherein said T regulatory cell secretes Galectin 9.

Preferred methods are drawn to embodiments wherein said T regulatory cell secretes IL-20.

Preferred methods are drawn to embodiments wherein said T regulatory cell expresses perforin.

Preferred methods are drawn to embodiments wherein said T regulatory cell expresses granzyme.

Preferred methods are drawn to embodiments wherein said T regulatory cell inhibits activation of a conventional T cell.

Preferred methods are drawn to embodiments wherein said conventional T cell does not express CD25.

Preferred methods are drawn to embodiments wherein said conventional T cell is activated by ligation of CD3.

Preferred methods are drawn to embodiments wherein said T conventional T cell is activated by ligation of CD3 and CD28.

Preferred methods are drawn to embodiments wherein said activation of said conventional T cell is proliferation.

Preferred methods are drawn to embodiments wherein said activation of said conventional T cell is cytokine secretion.

Preferred methods are drawn to embodiments wherein said cytokine is IL-2.

Preferred methods are drawn to embodiments wherein said cytokine is IL-4.

Preferred methods are drawn to embodiments wherein said cytokine is IL-6.

Preferred methods are drawn to embodiments wherein said cytokine is IL-10.

Preferred methods are drawn to embodiments wherein said cytokine is IL-12.

Preferred methods are drawn to embodiments wherein said cytokine is IL-7.

Preferred methods are drawn to embodiments wherein said cytokine is IL-13.

Preferred methods are drawn to embodiments wherein said cytokine is IL-15.

Preferred methods are drawn to embodiments wherein said cytokine is IL-18.

Preferred methods are drawn to embodiments wherein said immune cells are peripheral blood mononuclear cells.

Preferred methods are drawn to embodiments wherein said peripheral blood mononuclear cells are treated with an immune modulatory agent prior to contacting with said regenerative cells.

Preferred methods are drawn to embodiments wherein said immune modulatory agent is ultraviolet light.

Preferred methods are drawn to embodiments wherein said immune modulatory agent is HGF.

Preferred methods are drawn to embodiments wherein said immune modulatory agent is oxytocin.

Preferred methods are drawn to embodiments wherein said immune modulatory agent is NGF.

Preferred methods are drawn to embodiments wherein said immune modulatory agent is FGF-1.

Preferred methods are drawn to embodiments wherein said immune modulatory agent is FGF-2.

Preferred methods are drawn to embodiments wherein said immune modulatory agent is a toll like receptor activator.

Preferred methods are drawn to embodiments wherein oxytocin is administered to peripheral blood mononuclear cells in vitro for a period of 1 minute to 4 weeks.

Preferred methods are drawn to embodiments wherein oxytocin is administered to peripheral blood mononuclear cells in vitro for a period of 2 hours to 1 week.

Preferred methods are drawn to embodiments wherein oxytocin is administered to said fibroblasts in vitro for a period of 24 hours to 72 hours.

Preferred methods are drawn to embodiments wherein said oxytocin is administered at a concentration of 10 nM-10 uM.

Preferred methods are drawn to embodiments wherein said oxytocin is administered at a concentration of 100 nM-1 uM.

Preferred methods are drawn to embodiments wherein said immune modulatory agent is morphine.

Preferred methods are drawn to embodiments wherein said immune modulatory agent is curcumin.

Preferred methods are drawn to embodiments wherein said immune modulatory agent is TGF-beta.

Preferred methods are drawn to embodiments wherein said immune modulatory agent is galectin-1.

Preferred methods are drawn to embodiments wherein said immune modulatory agent is galectin-3.

Preferred methods are drawn to embodiments wherein said immune modulatory agent is galectin-9.

Preferred methods are drawn to embodiments wherein said immune modulatory agent is IL-1.

Preferred methods are drawn to embodiments wherein said immune modulatory agent is IL-2.

Preferred methods are drawn to embodiments wherein said immune modulatory agent is IL-4.

Preferred methods are drawn to embodiments wherein said immune modulatory agent is IL-7.

Preferred methods are drawn to embodiments wherein said immune modulatory agent is IL-10.

Preferred methods are drawn to embodiments wherein said immune modulatory agent is IL-13.

Preferred methods are drawn to embodiments wherein said immune modulatory agent is IL-15.

Preferred methods are drawn to embodiments wherein said immune modulatory agent is IL-12.

Preferred methods are drawn to embodiments wherein said immune modulatory agent is IL-18.

Preferred methods are drawn to embodiments wherein said immune modulatory agent is IL-20.

Preferred methods are drawn to embodiments wherein said immune modulatory agent is IL-22.

Preferred methods are drawn to embodiments wherein said immune modulatory agent is IL-35.

### DETAILED DESCRIPTION OF THE INVENTION

The invention provides means of increasing potency of TNF-alpha blockers as well as overcoming treatment resistance to TNF-alpha blockers. In one embodiment, the invention provides the utilization of autologous immune cells endowed with regenerative and immunomodulatory activity through exposure to factors generated by mesenchymal stem cell stimulated with factor resembling “danger” signals such as activators of the toll like receptor pathways.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.) and chemical methods.

The term positive, when used in the context of expression of a marker, refers to expression of an antigen assayed by a fluorescently labelled antibody, wherein the label's fluorescence on the structure (for example, a cell) referred to as “positive” is at least 30% higher (.gtoreq.30%), particularly .gtoreq.50% or .gtoreq.80%, in median fluorescence intensity in comparison to staining with an isotype-matched fluorescently labelled antibody which does not specifically bind to the same target. Such expression of a marker is indicated by a superscript “plus” (.sup.+), following the name of the marker, e.g. CD4.sup.+. If the word “expression” is used herein in the context of “gene expression” or “expression of a marker or biomolecule” and no further qualification of “expression” is mentioned, this implies “positive expression” as defined above.

In the present specification, the term negative, when used in the context of expression of a marker, refers to expression of an antigen assayed by a fluorescently labelled antibody, wherein the median fluorescence intensity is less than 30% higher, particularly less than 15% higher, than the median fluorescence intensity of an isotype-matched antibody which does not specifically bind the same target. Such expression of a marker is indicated by a superscript minus (.sup.−), following the name of the marker, e.g. CD25.sup.−.

Chemical Modification: As used herein, “chemical modification” refers to the process wherein a chemical or biochemical is used to induce genomic changes in the donor cell, or nucleus thereof, that allow the donor cell, or nucleus thereof, to be responsive during maturation and receptive to the host cell cytoplasm.

Committed: As used herein, “committed” refers to cells which are considered to be permanently committed to a specific function. Committed cells are also referred to as “terminally differentiated cells.”

Cytoplast Extract Modification: As used herein, “cytoplast extract modification” refers to the process wherein a cellular extract consisting of the cytoplasmic contents of a cell are used to induce genomic changes in the donor cell, or nucleus thereof, that allow the donor cell, or nucleus thereof, to be responsive during maturation and receptive to the host cell cytoplasm.

Dedifferentiation: As used herein, “dedifferentiation” refers to loss of specialization in form or function. In cells, dedifferentiation leads to an a less committed cell.

Differentiation: As used herein, “differentiation” refers to the adaptation of cells for a particular form or function. In cells, differentiation leads to a more committed cell.

Donor Cell: As used herein, “donor cell” refers to any diploid (2N) cell derived from a pre-embryonic, embryonic, fetal, or post-natal multi-cellular organism or a primordial sex cell which contributes its nuclear genetic material to the hybrid stem cell. The donor cell is not limited to those cells that are terminally differentiated or cells in the process of differentiation. For the purposes of this invention, donor cell refers to both the entire cell or the nucleus alone.

Donor Cell Preparation: As used herein, “donor cell preparation” refers to the process wherein the donor cell, or nucleus thereof, is prepared to undergo maturation or prepared to be receptive to a host cell cytoplasm and/or responsive within a post-natal environment.

Germ Cell: As used herein, “germ cell” refers to a reproductive cell such as a spermatocyte or an oocyte, or a cell that will develop into a reproductive cell.

Host Cell: As used herein, “host cell” refers to any multipotent stem cell derived from a pre-embryonic, embryonic, fetal, or post-natal multicellular organism that contributes the cytoplasm to a hybrid stem cell.

Host Cell Preparation: As used herein, “host cell preparation” refers to the process wherein the host cell is enucleated.

Hybrid Stem Cell: As used herein, “hybrid stem cell” refers to any cell that is multipotent and is derived from an enucleated host cell and a donor cell, or nucleus thereof, of a multicellular organism. Hybrid stem cells are further disclosed in co-pending U.S. patent application Ser. No. 10/864,788.

Karyoplast Extract Modification: As used herein, “karyoplast extract modification” refers to the process wherein a cellular extract consisting of the nuclear contents of a cell, lacking the DNA, are used to induce genomic changes in the donor cell, or nucleus thereof, that allow the donor cell, or nucleus thereof, to be responsive during maturation or receptive to the host cell cytoplasm.

Maturation: As used herein, “maturation” refers to a process of coordinated steps either forward or backward in the differentiation pathway and can refer to both differentiation or de-differentiation. As used herein, maturation is synonymous with the terms develop or development when applied to the process described herein.

Modified Germ Cell: As used herein, “modified germ cell” refers to a cell comprised of a host enucleated ovum and a donor nucleus from a spermatogonia, oogonia or a primordial sex cell. The host enucleated ovum and donor nucleus can be from the same or different species. A modified germ cell can also be called a “hybrid germ cell.”

Multipotent: As used herein, “multipotent” refers to cells that can give rise to several other cell types, but those cell types are limited in number. An example of a multipotent cells is hematopoietic cells-blood stem cells that can develop into several types of blood cells but cannot develop into brain cells.

Multipotent Adult Progenitor Cells: As used herein, “multipotent adult progenitor cells” refers to multipotent cells isolated from the bone marrow which have the potential to differentiate into mesenchymal, endothelial and endodermal lineage cells.

Pre-embryo: As used herein, “pre-embryo” refers to a fertilized egg in the early stage of development prior to cell division. During the pre-embryonic stage the initial stages of cleavage are occurring.

Pre-embryonic Stem Cell: See “Embryonic Stem Cell” above.

Post-natal Stem Cell: As used herein, “post-natal stem cell” refers to any cell that is multipotent and derived from a multi-cellular organism after birth.

Pluripotent: As used herein, “pluripotent” refers to cells that can give rise to any cell type except the cells of the placenta or other supporting cells of the uterus.

Primordial Sex Cell: As used herein, “primordial sex cell” refers to any diploid cell that is derived from the male or female mature or developing gonad, is able to generate cells that propagate a species and contains a diploid genomic state. Primordial sex cells can be quiescent or actively dividing. These cells include male gonocytes, female gonocytes, spermatogonial stem cells, ovarian stem cells, oogonia, type-A spermatogonia, Type-B spermatogonia. Also known as germ-line stem cells.

Primordial Germ Cell: As used herein, “primordial germ cell” refers to cells present in early embryogenesis that are destined to become germ cells.

Reprogamming: As used herein “reprogramming” refers to the resetting of the genetic program of a cell such that the cell exhibits pluripotency and has the potential to produce a fully developed organism.

Responsive: As used herein, “responsive” refers to the condition of a cell, or group of cells, wherein they are susceptible to and can function accordingly within a cellular environment. Responsive cells are capable of responding to and functioning in a particular cellular environment, tissue, organ and/or organ system.

Somatic Stem Cells: As used herein, “somatic stem cells” refers to diploid multipotent or pluripotent stem cells. Somatic stem cells are not totipotent stem cells. Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of multipotent cells is hematopoietic cells-blood cells that can develop into several types of blood cells, but cannot develop into brain cells. At the end of the long chain of cell divisions that make up the embryo are “terminally differentiated” cells-cells that are considered to be permanently committed to a specific function.

Therapeutic Cloning: As used herein, “therapeutic cloning” refers to the cloning of cells using nuclear transfer methods including replacing the nucleus of an ovum with the nucleus of another cell and stem cells derived from the inner cell mass.

Therapeutic Reprogramming: As used herein, “therapeutic reprogramming” refers to the process of maturation wherein a stem cell is exposed to stimulatory factors according to the teachings of the present invention to yield either pluripotent, multipotent or tissue-specific committed cells. Therapeutically reprogrammed cells are useful for implantation into a host to replace or repair diseased, damaged, defective or genetically impaired tissue. The therapeutically reprogrammed cells of the present invention do not possess non-human sialic acid residues.

Totipotent: As used herein, “totipotent” refers to cells that contain all the genetic information needed to create all the cells of the body plus the placenta. Human cells have the capacity to be totipotent only during the first few divisions of a fertilized egg.

Whole Cell Extract Modification: As used herein, “whole cell extract modification” refers to the process wherein a cellular extract consisting of the cytoplasmic and nuclear contents of a cell are used to induce genomic changes in the donor cell, or nucleus thereof, that allow the donor cell, or nucleus thereof, to be responsive during maturation and receptive to the host cell cytoplasm.

High expression of a marker, for example high expression of FoxP3, refers to the expression level of such marker in a clearly distinguishable cell population that is detected by FACS showing the highest fluorescence intensity per cell compared to the other populations characterized by a lower fluorescence intensity per cell. A high expression is indicated by superscript “high” or “hi” following the name of the marker, e.g. FoxP3.sup.high. The term “is expressed highly” refers to the same feature.

In one embodiment of the invention, mesenchymal stem cells are treated with 1-1000 IU of interferon gamma per ml, more preferably 20-500 IU of interferon gamma per ml, more preferably around 100 IU of interferon gamma per ml. Conditioned media is extracted after 1 to 96 hours of culture, more preferably after approximately 24 to 72 hours of culture, more preferably after approximately 48 hours of culture. In some embodiments of the invention conditioned media is generated under conditions of hypoxia. In some embodiments hypoxia is from 0.1%-10%, 0.1%-5%, 0.1%-2.5%, 0.1%-2%, 0.1%-1%, 0.5%-10%, 0.5%-7.5%, 0.5%-5%, 0.5%-2.5%, 0.5%-2%, 0.5%-1%, 1%-10%, 1%-7.5%, 1%-5%, 1%-2.5%, 1%-2%, 2%-10%, 2%-7.5%, 2%-5%, 2%-2.5%, 5%-10%, 5%-7.5%, 5%-6%, or 7.5%-10% oxygen; and/or the length of exposure to hypoxia is 30 minutes (min)-3 days, 30 min-2 days, 30 min-1 day, 30 min-12 hours (hrs), 30 min-8 hrs, 30 min-6 hrs, 30 min-4 hrs, 30 min-2 hrs, 30 min-1 hr, 1 hr-3 days, 1 hr-2 days, 1 hr-1 day, 1-12 hrs, 1-8 hrs, 1-6 hrs, 1-4 hrs, 1-2 hrs, 2 hrs-3 days, 2 hrs-2 days, 2 hrs-1 day, 2 hrs-12 hrs, 2-10 hrs, 2-8 hrs, 2-6 hrs, 2-4 hrs, 2-3 hrs, 6 hrs-3 days, 6 hrs-2 days, 6 hrs-1 day, 6-12 hrs, 6-8 hrs, 8 hrs-3 days, 8 hrs-2 days, 8 hrs-1 day, 8-16 hrs, 8-12 hrs, 8-10 hrs, 12 hrs-3, days, 12 hrs-2 days, 12 hrs-1 day, 12-18 hrs, 12-14 hrs, 1-3 days, or 1-2 days. Conditioned media is then added to a culture of immune cells autologous to the patient in need of treatment. In some embodiments the immune cells are CD4, in some said immune cells are CTLA-4 positive. In other embodiments, T cells that are characterized by expression of CD4 and CD25, and low expression of CD127 (CD4.sup.+CD25.sup.+CD127.sup.low) and/or by expression of both CD4 and FOXP3 (CD4.sup.+FOXP3.sup.+) are isolated in the tumour T cell isolation step, and subsequently sequenced exclusively. In certain embodiments, T cells with a T regulatory phenotype are characterized by markers known in the art including, without being limited to, CTLA-4, TIM3, GITR, LAG-3, CD69, TGF-beta, IL-10, particularly characterized by expression of CD4 and CD25

In one embodiment of the invention, are methods for producing a regenerative T cell by contacting a T cell with an effective amount of (i) one or more CD3 stimulation agent(s) in the absence of a CD28 stimulation agent for a first period of time under conditions that allow for stimulation and activation of the T cell. Then introducing into the T cell an effective amount of a nucleic acid sequence encoding a forkhead box protein 3 (FOXP3) polypeptide, where the presence of the nucleic acid sequence in the T cell induces the T cell to develop or further develop one or more characteristics of a T regulatory cell phenotype compared to when the nucleic acid sequence is not present in the T cell. In some embodiments, the method further comprises contacting the T cell with an effective amount of one or more agent(s) that decreases CD28 expression and/or activity during the first period of time. This is a process disclosed in the invention together with conducting this process in the presence of mesenchymal stem cells in a contact dependent and/or contact independent manner. In some embodiments, provided herein are methods of producing T regulatory cells by: contacting a T cell with an activated mesenchymal stem cell, wherein said mesenchymal stem cell is contacted with an activator of the TLR pathway and an effective amount of (i) one or more CD3-stimulation agent(s), and (ii) one or more CD28-stimulation agent(s) for a first period of time under conditions that allow for stimulation and activation of the T cell; contacting the T cell with an effective amount of one or more agent(s) that decreases CD28 expression and/or activity; and introducing into the T cell an effective amount of a nucleic acid sequence encoding a forkhead box protein 3 (FOXP3) polypeptide, wherein the presence of the nucleic acid sequence in the T cell induces the T cell to develop or further develop one or more characteristics of a T regulatory cell phenotype compared to when the nucleic acid sequence is not present in the T cell. In some embodiments, the step of contacting the T cell with an effective amount of one or more agent(s) that decreases CD28 expression and/or activity can be performed during the first period of time. In some embodiments, provided herein are methods for producing a T regulatory cell by contacting a T cell with an effective amount of (i) one or more CD3 stimulation agent(s) and (ii) one or more CD28 stimulation agent(s) for a first period of time under conditions that allow for stimulation and activation of the T cell, and (iii) one or more agent(s) that decreases CD28 expression and/or activity; and introducing into the T cell an effective amount of a nucleic acid sequence encoding a forkhead box protein 3 (FOXP3) polypeptide, where the presence of the nucleic acid sequence in the T cell induces the T cell to develop or further develop one or more characteristics of a T regulatory cell phenotype compared to when the nucleic acid sequence is not present in the T cell. Also provided herein are methods for producing a T cell that further includes contacting the T cell with an effective amount of interleukin-2 (IL-2) and/or TGF−.beta. for a second period of time under conditions that allow for stabilization of a T regulatory phenotype as compared to when the T cell is not contacted with IL-2 and/or TGF−.beta. for the second period of time, however this process is carried out in the presence of mesenchymal stem cells and/or conditioned media of mesenchymal stem cells.

The invention provides novel stem cell types, methods of manufacture, and therapeutic uses. Provided are means of deriving stem cells possessing regenerative, immune modulatory, anti-inflammatory, and angiogenic/neurogenic activity from umbilical cord tissue such as Wharton's Jelly. In some embodiments manipulation of stem cell “potency” is disclosed through hypoxic manipulation, growth on non-xenogeneic conditions, as well as addition of epigenetic modulators.

The cells of the invention are cultured under hypoxia, in one embodiment, cultured in order to induce and/or augment expression of chemokine receptors. One such receptor is CXCR-4. The population of cells, including population of umbilical cord mesenchymal cells, may be enriched for CXCR-4, such as (or such as about) 50%, 55%, 60%, 65%, 70%, 75%, 80%, 85%, 90%, 95%, 96%, 97%, 98%, 99%, or more of the population expressing CXCR-4, CD31, CD34, or any combination thereof. In addition, or alternatively, <1%, <2%, <3%, <4%, <5%, <6%, <7%, <8%, <9%, or <10% of the population of cells may express CD14 and/or CD45. The umbilical cord cells of the invention may further possess markers selected from the group consisting of STRO-1, CD105, CD54, CD56, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, SSEA-4, NANOG−, OCT-4+, and a combination thereof. In some embodiments said placental cells of the invention are admixed with endothelial cells. Said endothelial cells may express one or more markers selected from the group consisting of: a) extracellular vimentin; b) CD133; c) c-kit; d) VEGF receptor; e) activated protein C receptor; and f) a combination thereof. In some embodiments, the population of endothelial cells comprises endothelial progenitor cells.

The population of cells may be allogeneic, autologous, or xenogenic to an individual, including an individual being administered the population of cells. In some embodiments, the population of cells are matched by mixed lymphocyte reaction matching.

In some embodiments, the population of cells is derived from tissue selected from the group consisting of the placental body, placenta, umbilical cord tissue, adipose tissue, peripheral blood, hair follicle, cord blood, Wharton's Jelly, menstrual blood, endometrium, skin, omentum, amniotic fluid, and a combination thereof. In some embodiments, the population of cells, the population of umbilical mesenchymal stem cells, or the population of endothelial cells comprises human umbilical cord derived adherent cells. The human umbilical cord derived adherent cells may express a cytokines selected from the group consisting of: a) FGF-1; b) FGF-2; c) HGF; d) interleukin-1 receptor antagonist; and e) a combination thereof. In some embodiments, the population of cells, the population of umbilical cord cells express arginase, indoleamine 2,3 deoxygenase, interleukin-10, and/or interleukin 35. In some embodiments, the population of cells, the population of umbilical cord cells, or the population of endothelial cells express hTERT and Oct-4 but does not express a STRO-1 marker.

In some embodiments, the population of cells, or the population of umbilical cord cells has an ability to undergo cell division in less than 36 hours in a growth medium. In some embodiments, the population of cells, or the population of umbilical cord cells has an ability to proliferate at a rate of 0.9-1.2 doublings per 36 hours in growth media. In some embodiments, the population of cells, or the population of umbilical cord cells has an ability to proliferate at a rate of 0.9, 1.0, 1.1, or 1.2 doublings per 36 hours in growth media. The population of cells, or population of umbilical cord cells may produce exosomes capable of inducing more than 50% proliferation when the exosomes are cultured with human umbilical cord endothelial cells. The induction of proliferation may occur when the exosomes are cultured with the human umbilical cord endothelial cells at a concentration of 10, 20, 30, 40, 50, 60, 70, 80, 90, 100, 110, 120, 130, 140, 150, 160, 170, 180, 190, 200, or more exosomes per cell.

In some embodiments, a population of cells is generated by culture of recipient derived cells together with mesenchymal stem cells. One type of mesenchymal stem cells including a population of umbilical cells alone, are administered to an individual, including an individual having an acute or chronic pathology, wherein the population of cells may be administered via any suitable route, including as non-limiting examples, intramuscularly and/or intravenously. In some embodiments, a population of umbilical cord cells is optionally obtained, the population is then optionally contacted via culturing with a population of progenitor for T regulatory cells, wherein the culturing conditions allow for the generation of T regulatory cells. These generated T regulatory cells are then administered to an individual. In one embodiment, the invention teaches that phenotypically defined MSCs can be isolated from the Wharton's jelly of umbilical cord segments and defined morphologically and by cell surface markers. By dissecting out the veins and arteries of the cord segments and exposing the Wharton's jelly, the cells of invention, of one embodiment of the invention, may be obtained. An approximately 1-5 cm cord segment is placed in collagenase solution (1 mg/ml, Sigma) for approximately 18 hours at room temperature. After incubation, the remaining tissue is removed and the cell suspension is diluted with PBS into two 50 ml tubes and centrifuged. Cells are then washed in PBS and counted using hematocytometer. 5-20.times.10.sup.6 cells were then plated in a 6 cm tissue culture plate in low-glucose DMEM (Gibco) with 10% FBS (Hyclone), 2 mM L-Glutamine (Gibco), 100 U/ml penicillin/100 ug/ml streptomycin/0.025 ug/ml amphotericin B (Gibco). At this step of the purification process, cells are exposed to hypoxia. The amount of hypoxia needed is the sufficient amount to induce activation of HIF-1 alpha. In one embodiment cells are cultured for 24 hours at 2% oxygen. After 48 hours, cells are washed with PBS and given fresh media. Cells are given new media twice weekly. After 7 days, cells are approximately 70-80% confluent and are passed using HyQTase (Hyclone) into a 10 cm plate. Cells are then regularly passed 1:2 every 7 days or upon reaching 80% confluence.

In another embodiment of the invention, biologically useful stem cells are disclosed, of the mesenchymal or related lineages, which are therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover, the therapeutically reprogrammed cells of the present invention are immunologically privileged and therefore suitable for therapeutic applications. Additional methods of the present invention provide for the generation of hybrid stem cells. Furthermore, the present invention includes related methods for maturing stem cells made in accordance with the teachings of the present invention into specific host tissues. For use in the current invention, the practitioner is thought that ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied.

During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G0/G1 phase of the cell cycle. In mice and rats, these gonocytes resume division within a few days after birth to generate spermatogonial stem cells and eventually undergo differentiation and meiosis related to spermatogenesis. It is known that embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has led to the generation and differentiation of a wide range of cell types, including cardiomyocytes, hematopoietic cells, endothelial cells, nerves, skeletal muscle, chondrocytes, adipocytes, liver and pancreatic islets.

Growing embryonic stem cells in co-culture with mature cells can influence and initiate the differentiation of the embryonic stem cells to a particular lineage. Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive postnatally. Developmental processes are highly conserved amongst the different species allowing maturation or differentiation systems from one mammalian species to be extended to other mammalian species in the laboratory.

During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be effected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells.

Telomeres are the physical ends of chromosomes that contain highly conserved, tandemly repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress. In one teaching or embodiment, of the invention, therapeutically reprogrammed cells, in some embodiments mesenchymal stem cells, are provided. Therapeutic reprogramming refers to a maturation process wherein a stem cell is exposed to stimulatory factors according the teachings of the present invention to yield enhanced therapeutic activity. In some embodiments, enhancement of therapeutic activity may increase proliferation, in other embodiments, it may enhance chemotaxis. Other therapeutic characteristics include ability to under resistance to apoptosis, ability to overcome senescence, ability to differentiate into a variety of different cell types effectively, and ability to secrete therapeutic growth factors which enhance viability/activity, of endogenous stem cells.

In order to induce therapeutic reprogramming of cells, in some cases, as disclosed herein, of Wharton's jelly originating cells, the invention teaches the utilization of stimulatory factors, including without limitation, chemicals, biochemicals and cellular extracts to change the epigenetic programming of cells. These stimulatory factors induce, among other results, genomic methylation changes in the donor DNA. Embodiments of the present invention include methods for preparing cellular extracts from whole cells, cytoplasts, and karyoplasts, although other types of cellular extracts are contemplated as being within the scope of the present invention. In a non-limiting example, the cellular extracts of the present invention are prepared from stem cells, specifically embryonic stem cells. Donor cells are incubated with the chemicals, biochemicals or cellular extracts for defined periods of time, in a non-limiting example for approximately one hour to approximately two hours, and those reprogrammed cells that express embryonic stem cell markers, such as Oct4, after a culture period are then ready for transplantation, cryopreservation or further maturation.

In another embodiment of the present invention, hybrid stem cells are provided which can be used for cellular regenerative/reparative therapy. The hybrid stem cells of the present invention are pluripotent and customized for the intended recipient so that they are immunologically compatible with the recipient. Hybrid stem cells are a fusion product between a donor cell, or nucleus thereof, and a host cell. Typically, the fusion occurs between a donor nucleus and an enucleated host cell. The donor cell can be any diploid cell, including but not limited to, cells from pre-embryos, embryos, fetuses and post-natal organisms. More specifically, the donor cell can be a primordial sex cell, including but not limited to, oogonium or differentiated or undifferentiated spermatogonium, or an embryonic stem cell. Other non-limiting examples of donor cells are therapeutically reprogrammed cells, embryonic stem cells, fetal stem cells and multipotent adult progenitor cells. Preferably the donor cell has the phenotype of the intended recipient. The host cell can be isolated from tissues including, but not limited to, pre-embryos, embryos, fetuses and post-natal organisms and more specifically can include, but is not limited to, embryonic stem cells, fetal stem cells, multipotent adult progenitor cells and adipose-derived stem cells. In a non-limiting example, cultured cell lines can be used as donor cells. The donor and host cells can be from the same individual or different individuals. In one embodiment of the present invention, lymphocytes are used as donor cells and a two-step method is used to purify the donor cells. After the tissues are disassociated, an adhesion step will be performed to remove any possible contaminating adherent cells followed by a density gradient purification step. The majority of lymphocytes are quiescent (in G0 phase) and therefore can have a methylation status that conveys greater plasticity for reprogramming. Multipotent or pluripotent stem cells or cell lines useful as donor cells in embodiments of the present invention, are functionally defined as stem cells by their ability to undergo differentiation into a variety of cell types including, but not limited to, adipogenic, neurogenic, osteogenic, chondrogenic and cardiogenic cell.

In some embodiments, host cell enucleation for the generation of hybrid stem cells according to the teachings of the present invention can be conducted using a variety of means. In a non-limiting example, ADSCs were plated onto fibronectin coated tissue culture slides and treated with cells with either cytochalasin D or cytochalasin B. After treatment, the cells can be trypsinized or TrypLE, re-plated and are viable for about 72 hours post enucleation. Host cells and donor nuclei can be fused using one of a number of fusion methods known to those of skill in the art, including but not limited to electrofusion, microinjection, chemical fusion or virus-based fusion, and all methods of cellular fusion are envisioned as being within the scope of the present invention. The hybrid stem cells made according to the teachings of the present invention possess surface antigens and receptors from the enucleated host cell but has a nucleus from a developmentally younger cell. Consequently, the hybrid stem cells of the present invention will be receptive to cytokines, chemokines and other cell signaling agents, yet possess a nucleus free from age-related DNA damage. The therapeutically reprogrammed cells and hybrid stem cells made in accordance with the teachings of the present invention are useful in a wide range of therapeutic applications for cellular regenerative/reparative therapy. For example, and not intended as a limitation, the therapeutically reprogrammed cells and hybrid stem cells of the present invention can be used to replenish stem cells in animals whose natural stem cells have been depleted due to age or ablation therapy such as cancer radiotherapy and chemotherapy. In another non-limiting example, the therapeutically reprogrammed cells and hybrid stem cells of the present invention are useful in organ regeneration and tissue repair. In one embodiment of the present invention, therapeutically reprogrammed cells and hybrid stem cells can be used to reinvigorate damaged muscle tissue including dystrophic muscles and muscles damaged by ischemic events such as myocardial infarcts. In another embodiment of the present invention, the therapeutically reprogrammed cells and hybrid stem cells disclosed herein can be used to ameliorate scarring in animals, including humans, following a traumatic injury or surgery. In this embodiment, the therapeutically reprogrammed cells and hybrid stem cells of the present invention are administered systemically, such as intravenously, and migrate to the site of the freshly traumatized tissue recruited by circulating cytokines secreted by the damaged cells. In another embodiment of the present invention, the therapeutically reprogrammed cells and hybrid stem cells can be administered locally to a treatment site in need of repair or regeneration.

In one embodiment, umbilical cord samples were obtained following the delivery of normal term babies with Institutional Review Board approval. A portion of the umbilical cord was then cut into approximately 3 cm long segments. The segments were then placed immediately into 25 ml of phosphate buffered saline without calcium and magnesium (PBS) and 1.times. antibiotics (100 U/ml penicillin, 100 ug/ml streptomycin, 0.025 ug/ml amphotericin B). The tubes were then brought to the lab for dissection within 6 hours. Each 3 cm umbilical cord segment was dissected longitudinally utilizing aseptic technique. The tissue was carefully undermined and the umbilical vein and both umbilical arteries were removed. The remaining segment was sutured inside out and incubated in 25 ml of PBS, 1.times. antibiotic, and 1 mg/ml of collagenase at room temperature. After 16-18 hours the remaining suture and connective tissue was removed and discarded. The cell suspension was separated equally into two tubes, the cells were washed 3.times. by diluting with PBS to yield a final volume of 50 ml per tube, and then centrifuged. Red blood cells were then lysed using a hypotonic solution. Cells were plated onto 6-well plates at a concentration of 5-20.times.10.sup.6 cells per well. UC-MSC were cultured in low-glucose DMEM (Gibco) with 10% FBS (Hyclone), 2 mM L-Glutamine (Gibco), 100 U/ml penicillin, 100 ug/ml streptomycin, 0.025 ug/ml amphotericin B (Gibco). Cells were washed 48 hours after the initial plating with PBS and given fresh media. Cell culture media were subsequently changed twice a week through half media changes. After 7 days or approximately 70-80% confluence, cells were passed using HyQTase (Hyclone) into a 10 cm plate. Cells were then regularly passed 1:2 every 7 days or upon reaching 80% confluence. Alternatively, 0.25% HQ trypsin/EDTA (Hyclone) was used to passage cells in a similar manner.

1. A method of overcoming resistance to TNF-alpha blockade in a patient with rheumatoid arthritis comprising the steps of: a) obtaining a sample of peripheral blood mononuclear cells from said patient; b) exposing said cells to conditioned media from a regenerative cell population; c) generating said conditioned media of “b” through activating said regenerative cell population; d) obtaining said patient peripheral blood mononuclear cells or isolated component cells thereof; e) optionally expanding said cells ex vivo; and f) administering said heterogenous or said homogeneous cell population of “e” into said patient in need of therapy.

2. The method of claim 1, wherein immune cells are isolated from peripheral blood mononuclear cells.

3. The method of claim 2, wherein said immune cells are T cells.

4. The method of claim 2, wherein said T cells are Th2 cells.

5. The method of claim 4, wherein said Th2 cells have a proclivity to produce more interleukin-4 than interferon gamma upon stimulation via CD3.

6. The method of claim 4, wherein said Th2 cells express GATA-3.

7. The method of claim 4, wherein said Th2 cells express IRF-4.

8. The method of claim 4, wherein said Th2 cells express CXCR4.

9. The method of claim 4, wherein said Th2 cells express interleukin-4 receptor.

10. The method of claim 4, wherein said Th2 cells express interleukin-33 receptor.

11. The method of claim 3, wherein said T cells are Th9 cells.

12. The method of claim 11, wherein said Th9 cell produces interleukin-9.

13. The method of claim 11, wherein said Th9 cell expresses IRF4.

14. The method of claim 11, wherein said Th9 cell secretes IL-10.

15. The method of claim 11, wherein said Th9 cell expresses TGF-beta receptor II.

16. The method of claim 1, wherein said regenerative cell population is a pluripotent stem cell.

17. The method of claim 16, wherein said pluripotent stem cell is an inducible pluripotent stem cell.

18. The method of claim 3, wherein said T cell is a T regulatory cell.

19. The method of claim 18, wherein said T regulatory cell expresses IL-7 receptor.

20. The method of claim 18, wherein said T regulatory cell expresses folate receptor.
